(19) United States
(12) Patent Application Publication
(10) Pub. No.: US 2015/0367081 A1
(43) Pub. Date: Dec. 24, 2015

(54) DISPENSER FOR A MEDICATION

(71) Applicant: ELI LILLY AND COMPANY, Indianapolis, IN (US)

(72) Inventor: Gerald Norman Rosenthal, Cape Town (ZA)

(21) Appl. No.: 14/764,989

(22) PCT Filed: Dec. 19, 2013
(86) PCT No.: PCT/IB2013/061136

§ 371 Date: Jul. 31, 2015

(30) Foreign Application Priority Data
Feb. 5, 2013 (ZA) 2013/00942

(51) Int. Cl. A61M 5/315; A61M 5/31

(52) U.S. Cl. CPC A61M 5/31568; A61M 5/3129; A61M 5/31591; A61M 2205/583; A61M 2005/3126

(57) ABSTRACT

A dispenser (10, 10.1) for medication is disclosed which dispenser comprises a marker (18, 24) and a set of indicia, the indicia being recognizable by 10 the user of the dispenser as indicating meals that are taken during each day. The marker and indicia are displaceable with respect to one another so that the juxtaposition of a specific indicium and the marker conveys to the user of the dispenser the meal at which the last administration of medication took place. The indicia and the marker are one on a barrel (12, 12.1) of the dispenser and the other on a cap (14, 14.1) of the dispenser. The cap has a discrete number of positions in which it can be fitted to the barrel.

### FIELD OF THE INVENTION

THIS INVENTION relates to a dispenser for a medication such as insulin.

### BACKGROUND TO THE INVENTION

To enable those suffering from diabetes to dose themselves with insulin it is provided in dispensers which, when operated, provide a measured dosage that is intended to maintain the blood sugar level within an acceptable range for a predetermined period of time.

A difficulty that users have is that, sometime after dosing, they are not always certain that they have dosed. If they should incorrectly dose again, this can cause hypoglycaemia which can lead to death. Missed doses can also result in serious undesirable medical outcomes.

U.S. Pat. No. 5,645,534 (Becton Dickinson) discloses a medication delivery pen which is constructed to allow a cap of the pen to be secured to a body of the pen in any one of fourteen different positions. Indicia spaced apart around the body of the pen represent days of the week. The indicia shown is constituted by the initial letters of the days of the week, that is, SMTWTFS.

The cap carries the indications “AM.” and “PM.” as well as an arrow relating to each of these times of the day.

When the cap is refitted to the body after medication has been administered to, or self administered by, the patient, the cap is positioned so that the AM arrow or the PM arrow points to the letter representing the day on which administration has just taken place. That one of the two arrows which points to that letter indicates whether the medicament was administered in the morning or in the evening.

When the pen is next looked at, the day of the last administration and whether it was undertaken in the morning or the evening is immediately apparent.

The specification does foreshadow that more or less than fourteen different discrete relative cap and body positions are possible. It does not, however, deal with why or when such a modification to a different number of discrete relative positions would be desirable.

The pen of the United States specification consequently allows for two administrations per day, one in the morning and one in the evening, to be recorded by the pen.

Subsequent to the date of issue of the Becton Dickinson patent, an alternative regimen for delivery of insulin has become widely used. This new regimen, termed basal-bolus, although invented in 1972 (by Dr Richard K. Bernstein), was not widely exploited until development, more than 20 years later, of two insulin analogs:
rapid-acting lispro (brand name Humalog) by Eli Lilly (approved 1996) and
long-acting glargine (brand name Lantus) by Sanofi-aventis (approved 2000).

The basal-bolus regimen, which includes an injection at each meal, attempts roughly to emulate how a non-diabetic person's body delivers insulin.

The essential merit of the rapid-acting analog insulin is that it is absorbed, peaks and breaks down much more quickly than the regular insulin that was used previously. For patients using the basal-bolus regimen of long-acting and rapid-acting analog insulins, the administration of insulin has thus become a meal-related event and not a time-related event as embodied in the Becton Dickinson patent.

The pen of the U.S. Pat. No. 5,645,534 is not able to provide the information that a patient using the basal-bolus regimen needs to ensure accurate control of the dosing regimen to avoid the potential medical difficulties discussed above. The present invention provides a dispenser which includes means for unambiguously indicating that the scheduled meal-related basal-bolus dosage has been administered.

### BRIEF DESCRIPTION OF THE INVENTION

According to one aspect of the present invention there is provided a dispenser for a medication which dispenser comprises a marker and a set of indicia, the indicia being recognizable by the user of the dispenser as indicating meals that are taken during each day, and the marker and indicia being displaceable with respect to one another so that the juxtaposition of a specific indicium and the marker conveys to the user of the dispenser the meal at which the last administration of medication took place.

According to a further aspect of the present invention there is provided a dispenser for medication which dispenser comprises a set of indicia and a marker, the indicia and the marker being displaceable relatively to one another, each indicium designating a meal-related time of day, or a day of the week, selected in dependence on the regimen being followed by the dispenser's user, and the position of the marker with respect to the indicia conveying to the user of the dispenser information as to the time or as to the day of the last administration of medication, the set of indicia including an indicium which is additional to the time-based or day-based indicia and which can be used, by associating the marker with it, to record that a dosage of medication additional to the regular dosages of the regimen being followed has been administered.

The indicia and the marker can be one on a barrel of the dispenser and the other on a cap of the dispenser, the cap having a discrete number of positions in which it can be fitted to the barrel.

In a further form the indicia and the marker are one on a rotatable ring and the other on a part with respect to which the ring can be rotated. The ring can be fitted to a main part of the cap or to a main part of the barrel.

### BRIEF DESCRIPTION OF THE DRAWINGS

For a better understanding of the present invention, and to show how the same may be carried into effect, reference will now be made, by way of example, to the accompanying drawings in which;

FIG. 1 is a pictorial view of a first form of dispenser in accordance with the present invention;

FIGS. 2a, 2b, 2c illustrate indicia that can be applied to the dispenser;

FIG. 3 is a pictorial view of a second form of insulin dispenser; and

FIG. 4 is a pictorial view of a third form of dispenser.

### DETAILED DESCRIPTION OF THE DRAWINGS

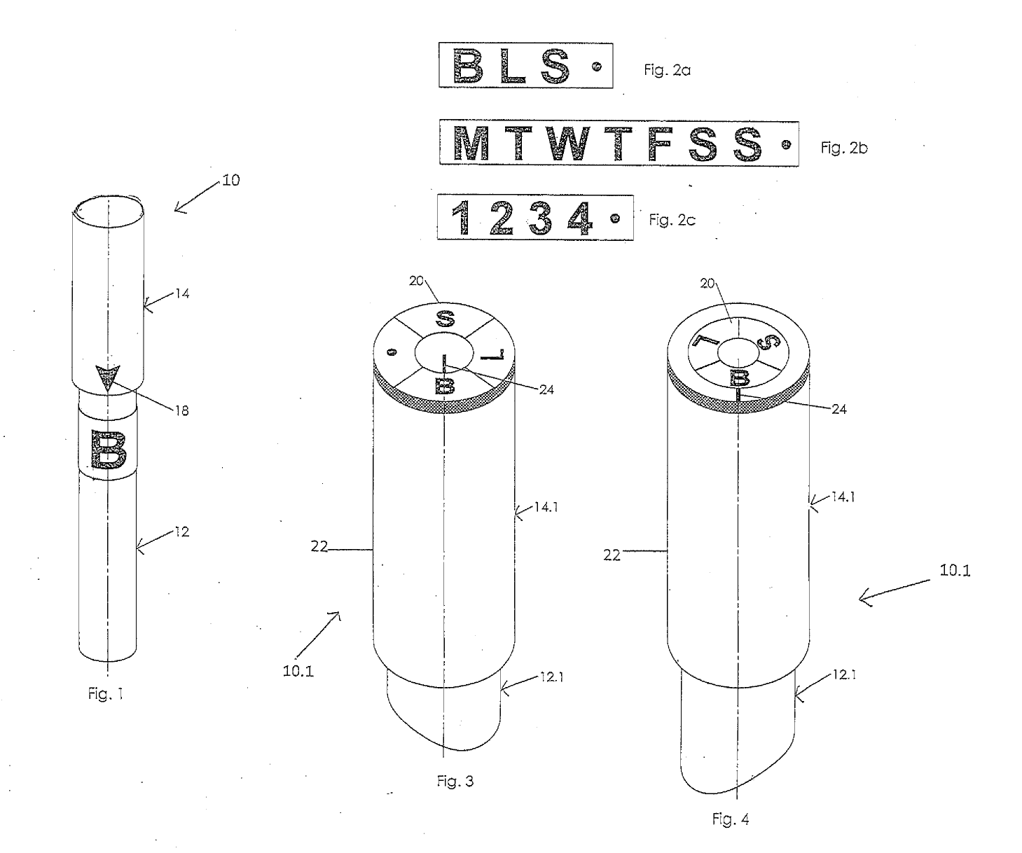

The dispenser 10 shown in FIG. 1 comprises a barrel 12 and a cap 14. The insulin or other medication to be dispensed is contained in the barrel 12. The mechanism which dispenses a dose of insulin from the barrel is not illustrated and will not be described as it forms no part of the present invention.

The cap 14 is formed with a readily visible marker. In the illustrated form it is constituted by an integrally moulded arrow head 18 which stands proud of the surface of the cap and has its tip adjacent the open end of the cap 14. The integrally moulded marker can be replaced by a separate element so that it can be of a different colour to the cap.

The cap 12 and barrel 14 are formed so that the cap can be fitted to the barrel in three different discrete positions. This can be achieved, for example, by a combination of three axially extending grooves and one axially extending spline, or three splines and one axially extending groove. As will be understood from the following description, the number of splines and grooves can be increased.

The barrel 12 carries one of the indicia illustrated in FIGS. 2a, 2b and 2c representing the meal-related time at which, or the day on which, a dosage is to be administered. These indicia can be moulded into the barrel. Alternatively they can be on a label which is affixed to the barrel or on a sleeve which fits around the barrel.

The indicia of FIG. 2a comprise the letters B, L and S standing for breakfast, lunch and supper and hence these indicia are meal-related. The letter D designating dinner could be substituted for S. In a further modification the letters D, T and BT can be used designating dinnertime, teatime and bedtime. If there is sufficient space the entire word and not just its initial, can be used. It will be understood that for speakers of languages other than English the letters referred to in this paragraph will be meaningless. For other languages the letters used will depend on the words that denote meals. Alternatively the letters can be replaced by graphic icons which will be understood as pertaining to specific meal times.

A dispenser with the indicia of FIG. 2a is for use by a patient requiring three doses per day. After dosing in the morning the cap is re-secured to the barrel with the arrow pointing at B which means to the patient that the breakfast dosage has been dispensed.

In FIG. 2b the letters indicate the days of the week and is the form provided for a patient that administers a basal dose once per day. The arrow can be pointed, after use, at the indicium indicating the day for which a dosage has been administered. The number of positions in which the cap can be secured to the barrel has to be increased to seven in this embodiment.

FIG. 2c shows indicia for use if four doses a day are recommended for a specific patient. Four splines or four grooves co-operating with one groove or one spline are required in this embodiment.

The indicia can be adjacent the open end of the cap and the arrow can be on the barrel if desired.

In FIG. 3 the dispenser is designated 10.1, the barrel 12.1 and the cap 14.1. The cap has at one end, the end that is remote from the barrel, a ring 20 which is snap fitted to the main part 22 of the cap. The ring 20 cannot be removed but is rotatable with respect to the main part 22 of the cap. The main part 22 and the ring 20 are respectively provided with, for example, depressions or detents. The detents fit in the depressions. When a force is applied to rotate the ring 20, the detents ride-up out of the depressions in which they are seated and snap into the adjacent depressions.

In the illustrated form the ring 20 carries the indicia of FIG. 2a and the closed end of the cap's main part 22 carries a marker 24.

The components on which the indicia and the marker are provided can be reversed as shown in FIG. 4 where the marker 24 is carried by the ring 20 and the indicia are on the end surface of the main part of the cap. The indicia shown in FIGS. 3 and 4 can be replaced by the indicia of FIG. 2b or of FIG. 2c if the dispenser is to be used daily or four times per day. In a still further form the ring 20 is fitted to the barrel 12.1 instead of to the cap 14.1. The indicia can in this form again be on the ring or on the barrel.

In each of FIGS. 2a to 2c there is, adjacent the right hand side of the set of indicia, a “dot”. The dot can be replaced by any indicium which cannot be confused with the other indicia of the set of which it forms a part. If the “dot” is used then, in the embodiment of FIG. 1, the cap must be able to fit in the barrel in one more position than is required if the neutral “dot” indicium is not used. Likewise, in the embodiment of FIGS. 3 and 4, the ring must have an additional position to which it can be adjusted.

Patients are given a regimen to follow. Possible regimens are at meals (which would normally mean three or four times a day), or four times a day at specific times not necessarily related to meals, or once per day for a basal dose. FIGS. 2a, 2b and 2c cover all these possibilities.

The marker is aligned with the “dot” to show that a dosage in addition to the regular dosage of the regimen has been administered.

Whilst the dispenser illustrated is intended for use in administering a liquid medication, it could be adapted for use in delivering other forms of medication such as capsules and pills.

The method of use that is preferred by patients and medical practitioners is that the marker and indicia be positioned to show when a dosage was last administered. However, if the patient finds it more convenient the marker and indicia can be positioned to show when the next administration is due.

1. A dispenser for medication which dispenser comprises a marker and a set of indicia, the indicia being recognizable by the user of the dispenser as indicating meals that are taken during each day, and the marker and indicia being displaceable with respect to one another so that the juxtaposition of a specific indicium and the marker conveys to the user of the dispenser the meal at which the last administration of medication took place.

2. A dispenser for medication which dispenser comprises a set of indicia and a marker, the indicia and the marker being displaceable relatively to one another, each indicium designating a meal-related time of day, or a day of the week, selected in dependence on the regimen being followed by the dispenser's user, and the position of the marker with respect to the indicia conveying to the user of the dispenser information as to the time or as to the day of the last administration of medication, the set of indicia including an indicium which is additional to the time-based or day-based based indicia and which can be used, by associating the marker with it, to record that a dosage of medication additional to the regular dosages of the regimen being followed has been administered.

3. A dispenser as claimed in claim 1, wherein the indicia and the marker are one on a barrel of the dispenser and the other on a cap of the dispenser, the cap having a discrete number of positions in which it can be fitted to the barrel.

4. A dispenser as claimed in claim 1, wherein the indicia and the marker are one on a rotatable ring and the other on a part with respect to which the ring can be rotated.

5. A dispenser as claimed in claim 4, wherein the ring is fitted to a main part of the cap.

6. A dispenser as claimed in claim 4, wherein the ring is fitted to Moll a main part of the barrel.

7. A dispenser as claimed in claim 2, wherein the indicia and the marker are one on a barrel of the dispenser and the other on a cap of the dispenser, the cap having a discrete number of positions in which it can be fitted to the barrel.

8. A dispenser as claimed in claim 2, wherein the indicia and the marker are one on a rotatable ring and the other on a part with respect to which the ring can be rotated.

9. A dispenser as claimed in claim 8, wherein the ring is fitted to a main part of the cap.

10. A dispenser as claimed in claim 8, wherein the ring is fitted to a main part of the barrel.
